(19) United States
(12) Patent Application Publication
(10) Pub. No.: US 2022/0074935 A1
(43) Pub. Date: Mar. 10, 2022

(54) SYSTEMS AND METHODS OF DETERMINING HYGIENE CONDITION OF AN INTERIOR SPACE

(71) Applicant: The Procter & Gamble Company, Cincinnati, OH (US)

(72) Inventors: Jiquan LIU, Singapore (SG); Tze Hau LAM, Singapore (SG); He ZHAO, Singapore (SG); Rahul VYAS, Singapore (SG); Garima CHAUHAN, Singapore (SG); Jian XU, Qingdao (CN); Pengfei ZHU, Qingdao (CN)

(21) Appl. No.: 17/471,369

(22) Filed: Sep. 10, 2021

(30) Foreign Application Priority Data
Sep. 10, 2020 (CN) PCT/CN2020/114438

(51) Int. Cl. G01N 33/569; G01N 33/36

(52) U.S. Cl. CPC G01N 33/56911; G16B 5/00; G01N 33/367

(57) ABSTRACT

A computer-implemented method for determining a hygiene condition of an interior space is described. The method has the steps of: a) obtaining relative abundance of at least one bacterium of human health concern in an interior space; b) generating a Microbial Index of Interior Space (“Microbial Index”) for the interior space based on the relative abundance; and c) displaying an output indicative of the Microbial Index for determining a hygiene condition of the interior space. The Microbial Index is characterized by a function of the relative abundance of the at least one bacterium of human health concern defined by F(Σᵢ₌₁^{N}Pᵢ), wherein N: Number of bacteria of human health concern identified in a microbial community in the interior space; P: Relative abundance of i-th bacteria of human health concern in the microbial community.

### TECHNICAL FIELD

The present invention relates to systems and methods for processing information associated with microorganisms of human health concern on a target surface in an interior space. In particular, the present invention relates to a system and a method for determining hygiene condition of an interior space.

### BACKGROUND

Presence of microorganisms of human health concern and their metabolites on surfaces in an interior environment, clothing and/or on skin can lead to health risks including but not limited to exacerbating eczema, skin irritations, spread of respiratory and gastrointestinal infections and diseases.

Antimicrobial active agents, also commonly known in the antimicrobial industry as “antimicrobial actives”, have been widely used as ingredients in consumer products to inhibit prokaryotic or eukaryotic organisms (“microorganisms”). The consumer products may include household cleaning products, personal care or cleansing products, air freshener products, laundry care products and oral care products. The increase in popularity of such antimicrobial products may be attributed to an increase in consumer awareness of a need to preventing transmission of microorganisms in an interior environment, especially in the household environment.

An example of a standard in vitro method for assessing an antimicrobial condition of a textile is the Japanese Industrial Standard 1902 (“JIS L 1902 Standard”, or ISO 20743). The JIS L 1902 Standard specifies quantitative and qualitative test methods to determine the antibacterial activity of all antibacterial textile products including nonwovens and antibacterial efficacy.

However, such in vitro methods typically require to be carried out in a microbiology testing laboratory under specific experimental conditions, which do not necessarily reflect consumer product use conditions. Other factors affecting such evaluations include only a limited number of bacteria can be evaluated at each time, relative scarcity of suitable test bacteria. As the factors do not reflect normal consumer product use conditions, and such methods are also not accessible to the consumers for making an independent assessment. Further, there is a diverse variety of bacteria and it can be difficult for a lay person to understand all the different types of bacterium which impact human health based on information obtained from experimental results performed under the JIS L 1902 Standard.

Therefore, without access to a method that is easily understood, consumers are not able to assess a hygiene condition of surfaces with the unaided eye.

Accordingly, there is a need for a simple method of assessing a hygiene condition of an interior space so as to enable proactive hygiene practices at an earlier stage.

### SUMMARY

The present invention relates to a computer-implemented method for determining a hygiene condition of an interior space, the method comprising the steps of:
a) obtaining relative abundance of at least one bacterium of human health concern in an interior space;
b) generating a Microbial Index of Interior Space (“Microbial Index”) based on the relative abundance; and
c) displaying an output indicative of the Microbial Index for determining a hygiene condition of the interior space;

wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in the interior space;
P: Relative abundance of i-th bacteria of human health concern in the microbial community.

### BRIEF DESCRIPTION OF THE DRAWINGS

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate various embodiments described herein, and together with the description serve to explain the principles and operations of the claimed subject matter.

FIG. 1 is a diagram illustrating an exemplary system for determining a hygiene condition of an interior space over a network according to the present invention;

FIG. 2 is a flow chart illustrating a method for determining a hygiene condition of an interior space according to the present invention;

FIG. 3 is a diagram illustrating an alternative system for determining a hygiene condition of an interior space over a network according to the present invention;

FIG. 4 is a flow chart illustrating a method of obtaining raw sequencing data according to the present invention;

FIG. 5 is a diagram illustrating an exemplary functional block diagram of a server computing system for generating a Microbial Index according to the present invention;

FIG. 6 is a flow chart illustrating a method for generating a Microbial Index of an interior space according to the present invention;

FIG. 7 is a flow chart illustrating a method for analyzing raw sequence data of all bacteria in the microbial community prior to obtaining relative abundance;

FIG. 8 is a flow chart illustrating a method for obtaining relative abundance of at least one bacterium of human health concern according to the present invention;

FIG. 9 is a flow chart illustrating a method for generating a Microbial Index according to the present invention;

FIG. 10 is a flow chart illustrating a method for providing a hygiene assessment according to the present invention;

FIG. 11 is a flow chart illustrating a method for providing a product recommendation for improving a hygiene condition of an interior space according to the present invention;

FIG. 12 is a screen shot illustrating an exemplary graphical user interface presenting an output indicative of a Microbial Index to a user for visualizing a hygiene condition of an interior space according to the present invention;

FIG. 13 is a screen shot illustrating an exemplary graphical user interface presenting a product recommendation to a user for providing a product recommendation for improving a hygiene condition of an interior space according to the present invention;

FIG. 14 is a flow chart illustrating a method of demonstrating efficacy of an antibacterial consumer product according to the present invention;

FIG. 15 is a graph illustrating a Microbial Index for an interior space, the interior space is a built interior space;

FIG. 16 is a graph illustrating different Microbial indices for a plurality of interior spaces;

FIG. 17 is an antibacterial air freshener product evaluated in Example 3;

FIG. 18 is a photograph of a test environment for the experiment conducted in Example 3; and

FIG. 19 is a graph illustrating different Microbial Indices for a plurality of surfaces in an interior space, the interior space is a toilet.

### DETAILED DESCRIPTION

There are many microorganisms present on surfaces in an interior space or an interior environment, clothing and/or on skin. Microorganisms include moulds, yeasts, bacteria and viruses. However, not every bacterium is of human health concern. Most consumers are not equipped with tools to identify all possible relevant bacteria of human health concern found in their living spaces and built environment/surfaces to distinguish bacteria of human health concern from commensal bacteria. As a result, consumers may “overdose” on antibacterial products or do not apply sufficient products in efforts to clean the interior space and/or surfaces in the interior space.

The present invention relates to a method, server and system for determining a hygiene condition of an interior space, and a graphical user interface for visualizing a hygiene condition of an interior space. Specifically, the present invention relates to a computer-implemented method for determining a hygiene condition of an interior space, the method comprising the steps of:
a) obtaining relative abundance of at least one bacterium of human health concern in an interior space;
b) generating a Microbial Index of Interior Space (“Microbial Index”) based on the relative abundance; and
c) displaying an output indicative of a hygiene condition of the interior space based on the Microbial Index;

wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in the interior space;
P: Relative abundance of i-th bacteria of human health concern in the microbial community.
   Analysing relative abundance of at least one bacterium of human health concern and generating a Microbial Index based on the analysed relative abundance allows users to have an objective method for determining balance or presence of the bacterium of human health concern relative to a total amount of bacteria in the interior space.

As described herein, generating determining a hygiene condition of an interior space provides a benefit of enabling users to take preventive action to improve a hygiene condition in the interior space. In the following description, relative abundance of relative abundance of at least one bacterium of human health concern in the interior space is obtained by collecting and analyzing a microbiome community on at least one surface in the interior space. However, it is contemplated that the microbiome community may be obtained from air in the interior space by sucking of the air in the interior space using commercially available air collecting equipment. Examples of such commercially available air collecting equipment include:
Dust sampler: a vacuum fitted with DUSTREAM® collectors (indoor biotechnologies, Charlottesville, Va.) or an equivalent. Ashkaan K Fahimipour et al. mSystems, vol. 3 issue 6, 2018.
Air sampler: SASS 3100 air sampler
(https://www.resrchintl.com/SASS_3100_air_sampler.html)

Prior to describing the present invention in detail, the following terms are defined and terms not defined should be given their ordinary meaning as understood by a skilled person in the relevant art.

“Interior Space” as used herein means a built interior environment selected from the group consisting of: residential interior environment, a commercial interior environment, a vehicle interior environment; a space in the built interior environment; an equipment interior environment of an equipment selected from the group consisting of: a household appliance, a commercial appliance, interior environment, an appliance space in the household appliance is selected from the group consisting of: refrigerator, washing machine, automatic dishwashing machine, air conditioning system.

“Hygiene condition” as used herein means all physical conditions of an interior space that provide a cleanliness effect of the interior space and a probability of an effectiveness of the state of the interior space in terms of microbiome balance for prevention of illness and disease. Some non-limiting examples of a hygiene condition may include microbiome of interior environment of a new house prior to residential considered as a better hygiene condition compared with microbiome of the interior environment of a crowded subway station.

“Surface” as used herein means an inanimate surface selected from a group consisting of: vehicle interior surfaces, fabrics, carpets, built environment surfaces, household surfaces, the household surfaces is selected from a group consisting of: floors, walls, carpet padding, towels, curtains, wall paper, door-knot, phone, tablet, personal PC, TV set, audio set, game console, toys, books.

“Bacteria of human health concern” as used herein means bacteria that can cause human disease, including but not limited to Achromobacter xylosoxidans, Acinetobacter baumannii, Actinomyces israelii, Aeromonas species, Bacillus anthracis, Bacteroides fragilis, Bacteroides melaninogenicus, Bartonella species, Bordetella pertussis, Borrelia species, Brucella species, Burkholderia species, Campylobacter, Capnocytophaga species, Chlamydophila pneumoniae, Chlamydophila psittaci, Citrobacter species, Clostridium species, Corynebacterium species, Coxiella burnetii, Ehrlichia species, Eikenella corrodens, Enterobacter species, Enterococcus faecalis, Enterococcus faecium, Escherichia coli, Francisella tularensis, Fusobacterium necrophorum, Gardnerella vaginalis, Haemophilus species, Helicobacter Pylori, Klebsiella species, Lactobacillus species, Legionella species, Leptospira species, Listeria monocytogenes, Moraxella catarrhalis, Morganella species, Mycoplasma pneumonia, Neisseria species, Nocardia species, Pasteurella multocida, Peptostreptococcus species, Porphyromonas gingivalis, Propionibacterium acnes, Proteus species, Providencia species, Pseudomonas aeruginosa, Salmonella species, Serratia marcescens, Shigella species, Staphylococcus aureus, Staphylococcus saprophyticus, Stenotrophomonas maltophilia, Streptococcus agalactiae, Streptococcus anginosus group, Streptococcus pneumoniae, Streptococcus pyogenes, Treponema pallidum, Vibrio species.

“Relative abundance” as used herein means the percent composition of a species of a microorganism relative to the total number of microorganisms in a given microbial community.

“Microbial Index” as used herein means a probability value indicative of a hygiene condition of an interior space, a target surface in an interior space, or a comparison of a hygiene condition of a target surface to a hygiene condition of a control surface in the interior space based on relative abundances of at least one bacterium of human concern on the target and control surfaces. The Microbial Index may be a numerical value determined by a Microbial Index generating model described herein with reference to the flow chart of FIG. 6. Specifically, a target surface having a lower Microbial Index than a control surface with a higher Microbial Index means the target surface is in a better hygiene condition relative to the control surface. On the other hand, a higher value of the Microbial Index corresponds to a less hygienic condition. The Microbial Index may comprise a value from 0.0 to 1.0 or 0.00 to 1.00. For example, a value of 0 may be indicative that the target surface is in the highest level of a hygiene condition (extremely hygienic), while a value of 1.0 is indicative that the target surface is in the lowest level of hygiene condition (extremely unhygienic).

“Microbiome database” as used herein means a database which has over 15,000 metagenomic sequences and 220,000 16S rRNA DNA sequences and (ii) an associated class definition (e.g. levels of hygiene) based on a specified range of Microbial Index.

“Microbial community” as used herein refers to groups of microorganisms that share a common living space. The common living space may be, such as for example, an interior space.

“User” as used herein refers to a person who uses at least the features provided herein, including, for example, a device user, a product user, a system user, and the like.

“Module” as used herein can be associated with software, hardware, or any combination thereof. In some implementations, one or more functions, tasks, and/or operations of modules can be carried out or performed by software routines, software processes, hardware, and/or any combination thereof.

“Treat”, “Treating” as used herein refers to providing a product recommendation, customized instructions, use of a recommended product for improving a hygiene condition of an interior space.

In the following description, the system described is a system 10 for determining a hygiene condition of an interior space. Accordingly, the server 14 described is a server 14 for determining hygiene condition of an interior space. A system for providing a product recommendation to improve hygiene condition of an interior space is also described. Accordingly, positive and negative attributes of hygiene in an interior space relate to presence of bacteria of human health concern in the interior space as described hereinbefore, i.e. all bacteria that reside on inanimate surfaces and which impact human health. However, it is contemplated that the systems, server and the method may be configured for use in a variety of applications to determine hygiene condition of other surfaces, such as for example, animate surfaces including mammal skin, wherein mammal skin is from one or more body parts including but not limited to the body, hands, arms, legs, and facial features including the nose, skin, lips, eyes, combinations thereof.

### System

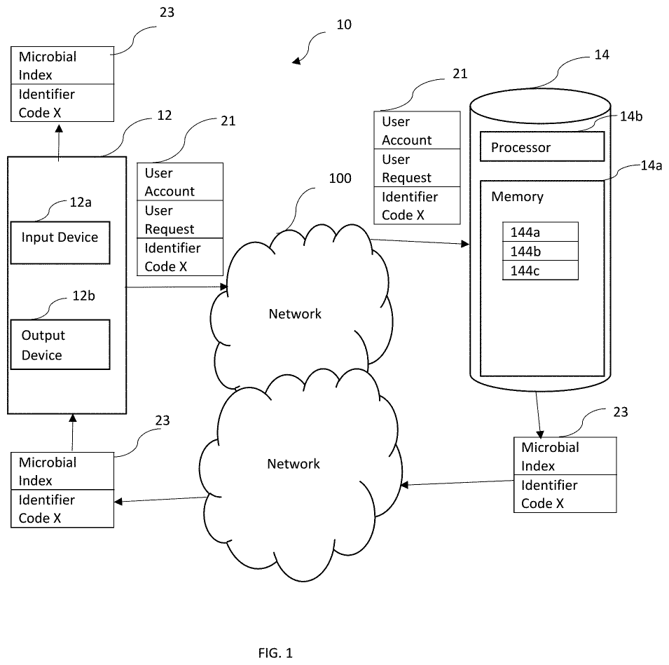

FIG. 1 is a schematic diagram illustrating a system 10 for determining hygiene condition of an interior space according to the present invention. In an exemplary embodiment, the system 10 is a cloud-based system configured for use anywhere, such as for example, through a portable electronic device 12.

The system 10 may include a network 100, which may be embodied as a wide area network (such as a mobile telephone network, a public switched telephone network, a satellite network, the internet, etc.), a local area network (such as wireless-fidelity, Wi-Max, ZigBee™, Bluetooth™ etc.), and/or other forms of networking capabilities. Coupled to the network 100 are the portable electronic device 12, and a server 14 for generating for display on a display, a graphical user interface for visualizing hygiene condition of an interior space. The server 14 is remotely located and connected to the portable electronic device 12 through the network 100. The network 100 may be used to acquire a user input 21 from the portable electronic device 12 and transmitting the user input 21 to the server 14 to be used in the method 101 according to the present invention described hereinafter with respect to FIG. 4. An input device 12a may be coupled to or integral with the portable electronic device 12 for receiving the user input 21 and an output device 12b for displaying an output 23 indicative of a hygiene condition of the interior space. The input device 12a may include but is not limited to a mouse, a touch screen display, or the like. The output device 12b may include but is not limited to a touch screen display, a non-touch screen display, a printer, audio output devices such as for example, speakers.

The user input 21 may be a user input request for determining a hygiene condition of the interior space. The user input 21 may be associated with a user account. If the user account is associated with a consumer product user, the user input 21 may comprise an user input request for a test kit for collecting microbiome from at least one surface in the interior space, the interior space is a residential interior environment, a commercial interior environment, a vehicle interior environment or a household appliance interior environment, wherein the household appliance is selected from the group consisting of: refrigerator, washing machine, automatic dishwashing machine, air conditioning system.

If the user account is associated with a lab user for analyzing relative abundance, the user input 21 may comprise relative abundance data comprising relative abundance of microorganisms in an interior space, particularly microorganisms on at least one surface in the interior space, more particularly, the relative abundance data may comprise relative abundance of at least one bacteria of human health concern on at least one surface in the interior space.

Specifically, the output 23 may be displayed in the graphical user interface. However, it is contemplated that the system 10 may be configured as a stand-alone system, and the output 23 may be displayed on a display connected to the stand-alone system. It is further contemplated that the portable electronic device 12 may be a touch sensitive display. The portable electronic device 12 may be a mobile telephone, a tablet, a laptop, a personal digital assistant and/or other computing device configured for capturing, storing, and/or transferring a user request 21 and/or relative abundance data. The portable electronic device 12 may also be configured for communicating with other computing devices via the network 100. The server 14 may include a non-transitory computer readable storage medium 14a (hereinafter “storage medium”), which stores relative abundance data obtaining logic 144a, Microbial Index generation logic 144b and graphic user interface (hereinafter “GUI”) logic 144c. The storage medium 14a may comprise random access memory (such as SRAM, DRAM, etc.), read only memory (ROM), registers, and/or other forms of computing storage hardware. The relative abundance obtaining logic 144a, Microbial Index generation logic 144b and the GUI logic 144c define computer executable instructions. A processor 14b is coupled to the storage medium 14a, wherein the processor 14b is configured to, based on the computer executable instructions, for implementing a method 101 for determining hygiene condition of an interior space according to the present invention as described hereinafter.

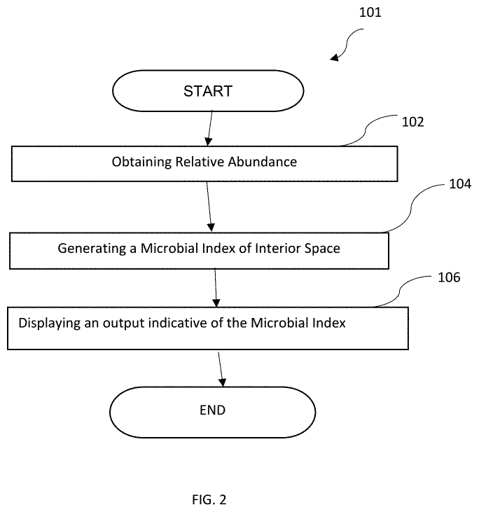

Accordingly, the steps 102, 104, 106 of the method 101 according to the present invention is described hereinafter with reference to FIG. 2 as individual processes for performing each step. Each process may also be described as a sub-routine, i.e. a sequence of program instructions that performs a corresponding step according to the method 101 according to the present invention.

When the processor 14b is initiated in response to a user input 21, the processor 14b causes relative abundance data to be obtained, e.g. via relative abundance obtaining logic 144a in step 102. The relative abundance data is a microorganism read count data structure as shown in Table 1 below.

TABLE 1

| | Identified as Bacterium | |
|---|---|---|
| Bacterium | of Human Health Concern | Reads Count |
| A | Yes | 50 |
| B | Yes | 25 |
| C | No | 100 |
| D | No | 100 |
| E | No | 75 |
| F | No | 25 |
| G | No | 125 |

The above read count data structure illustrates a list of identified microorganisms in a microbiome obtained from at least one surface in the interior space and read count of each of the identified microorganisms. Specifically, there are two bacteria of human health concern in the read count, in particular, Bacteria A and B are identified as bacteria of human health concern, while Bacteria C, D, E, F, G are not bacteria of human health concern.

In step 104, a Microbial Index is generated for the interior space based on the obtained relative abundance. The Microbial Index is characterized by a function of the relative abundance of the at least one bacterium of human health concern defined by F(Σᵢ₌₁^{N}Pᵢ), wherein
N: Number of bacteria of human health concern identified in a microbial community in the interior space;
P: Relative abundance of i-th bacteria of human health concern in the microbial community.

The function may be defined by the following formula:

$$Microbial Index of Interior Space = \frac{{\sum }_{i = 1}^{N} {P}_{i}}{{\sum }_{i = 1}^{M} {X}_{i}}$$

N: Number of bacteria of human health concern identified in a microbial community in the interior space
P: Relative abundance of i-th bacteria of human health concern in the microbial community
M: Number of all bacteria identified in a given microbiome
X: Relative abundance of i-th bacteria identified.

The method 101 may comprise displaying an output 23 indicative of the Microbial Index in step 106 to a user for determining hygiene condition of the interior space.

By generating a Microbial Index in response to a user request and displaying an output 23 indicative of the Microbial Index to the user, the user can obtain information related to a percentage of bacteria which impact human health to the total amount of bacteria on at least one surface in the interior space, thereby providing the user with a hygiene condition of the interior space in a concise and accurate manner that is easy to understand. It will be appreciated that the method 101 may also be adapted for application in relevant abundance data processing of other surfaces such as for example, animate surfaces including but limited to skin.

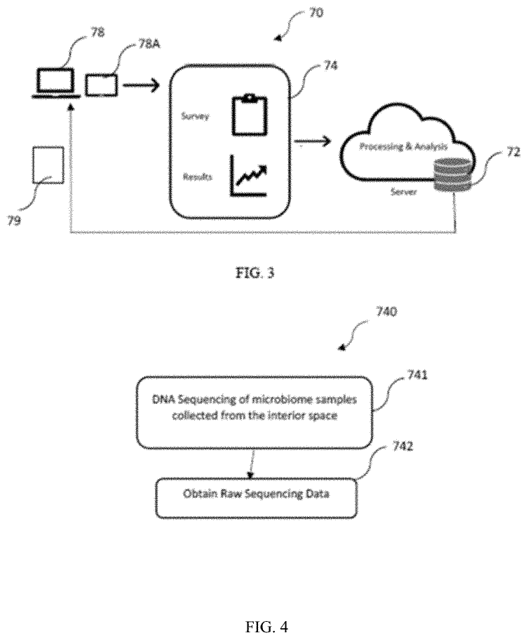

Details of how the Microbial Index is generated is described with respect to FIG. 3 and FIG. 4. FIG. 3 is a diagram illustrating an alternative system 70 for determining a hygiene condition of an interior space over a network according to the present invention. The system 70 comprises a web application capable of being compiled to run on a server computing system 72 for receiving a user input request 78A from a portable electronic device 78 for determining a hygiene condition of an interior space, wherein said server computing system 72 is in communication with a sequencing content analysis server 74 configured to store the received user input request 78A. The system 70 further comprises a display generating unit in communication with the server computing system (72), configured to display an output 79 indicative of the Microbial Index for the interior space on a portable electronic device 78 connected to the server through the network. As described hereinbefore, the Microbial Index is characterized by a function of the relative abundance of the at least one bacterium of human health concern defined by F(Σᵢ₌₁^{N}Pᵢ), wherein

N: Number of bacteria of human health concern identified in a microbial community;

P: Relative abundance of i-th bacteria of human health concern in the microbial community.

The sequencing content analysis server 74 may be configured for collecting and registering microbiome samples obtained from the interior space of the user who provided the user input request for the hygiene assessment. The server computing system 72, sequencing content analysis server 74 each illustrating an exemplary part of the system 70 cooperating with each other for providing the hygiene assessment to the portable electronic device 78 according to the present invention. Although the systems 72, 74 are described as a series of distributed systems which are processed in a sequential manner in response to a user request sent to the server computing system 72, it will be appreciated that the systems 72, 74 may be programmed in multiple ways to define an overall user interface for providing a hygiene assessment according to methods according to the present invention as described hereinbefore.

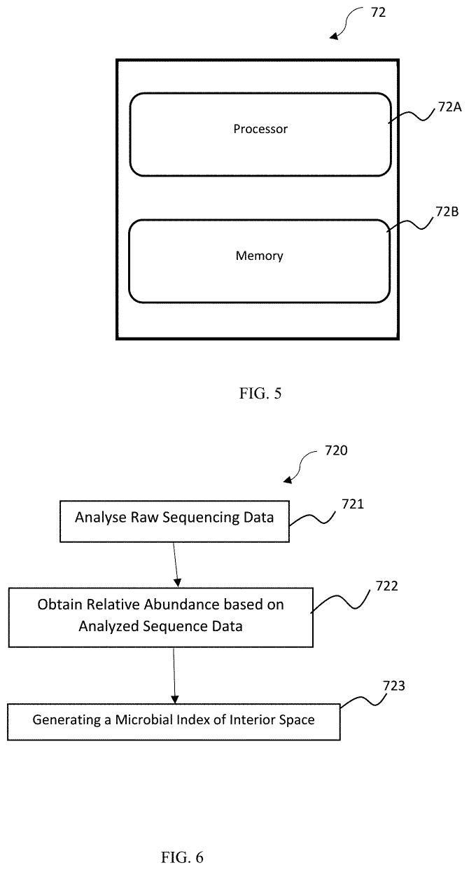

FIG. 4 is a flow chart illustrating a method 740 of obtaining raw sequencing data according to the present invention. Accordingly, the steps 741, 742 of the method 740 according to the present invention is described hereinafter with reference to FIG. 4 as individual processes for performing each step. Each process may also be described as a sub-routine, i.e. a sequence of program instructions that performs a corresponding step according to the method 740 according to the present invention. DNA from the surface samples can be determined by performing 16S rRNA sequencing or 2b-RAD Sequencing on the microbiome samples to obtain the DNA of the microorganisms present in the microbiome samples in step 741. In step 742, raw sequencing data is generated based on the extracted DNA and the raw sequencing data is analyzed for determining relative abundance of at least one bacteria of human health concern described hereinafter with reference to FIG. 5, and FIG. 6.

FIG. 5 is a diagram illustrating an exemplary functional block diagram of a server computing system 72 for generating a Microbial Index according to the present invention. The server computing system 72 comprises a server in communication with the web application through a network. The server comprises a processor 72A configured to, based on computer-executable instructions stored in a memory 72B to:
analyze raw sequencing data obtain a relative abundance of at least one bacterium of human health concern in the interior space; and
generate a Microbial Index of Interior Environment (“Microbial Index”) indicative of the hygiene condition of the interior space based on the relative abundance.

The processor 72A may comprise a raw sequence data analyzing module for analyzing raw sequence data and a Microbial Index generation module containing relative abundance data obtaining logic for obtaining relative abundance and microbial index generation logic for generating a Microbial Index for the interior space according to the present invention.

The raw sequence data analyzing module or the Microbial Index generation module may be implemented, in part or in whole, as software, hardware, or any combination thereof. In some cases, the Microbial Index generation module may be implemented, in part or in whole, as software running on one or more computing devices or computing systems, such as on a server computing system or a client computing system. For example, the Microbial Index generation module or at least a part thereof can be implemented as or within a mobile application (e.g. APP), a program or an applet, or the like, running on a client computing system such as the portable electronic device 12 of FIG. 1. The computing system may be in communication with a content server configured to store an obtained digital image or a plurality of obtained digital images. Each of the modules can be implemented using one or more computing devices or systems that include one or more servers, such as network servers or cloud servers.

FIG. 6 is a flow chart illustrating a method 720 for generating a Microbial Index of an interior space according to the present invention. Accordingly, the steps 721, 722, 723 of the method 720 according to the present invention is described hereinafter with reference to FIG. 5 as individual processes for performing each step. Each process may also be described as a sub-routine, i.e. a sequence of program instructions that performs a corresponding step according to the method 720 according to the present invention. The method 720 comprise substantially the steps of method 101 except that the method 720 comprises analyzing raw sequencing data in step 721 prior to obtaining relative abundance and generating a Microbial Index of an interior space. Specifically, in step 722, the relative abundance is obtained based on the analyzed sequence data and a Microbial Index of the interior space is generated based on the obtained relative abundance in step 723.

### Analyzing Raw Sequencing Data

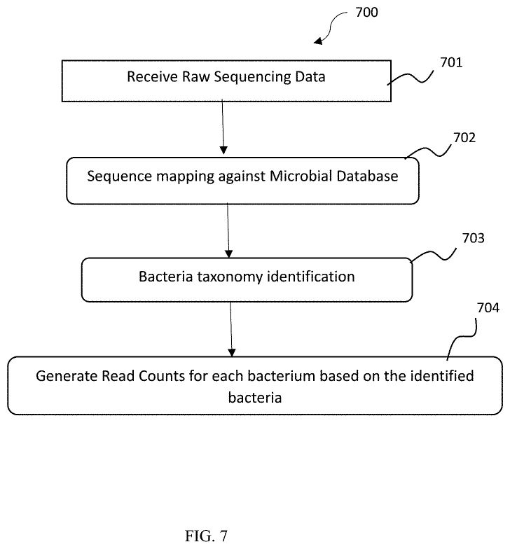

The step 721 of analyzing raw sequencing data of bacteria in a microbial community in the interior space according to the method 700 according to the present invention is described with reference to FIG. 7 which is a flow chart of a process of obtaining relative abundance corresponding to the step 721. The process 700 comprises receiving raw sequencing data of a microbial community on a target surface in the interior space in step 701. The raw sequencing data is mapped against content in a Microbial Database in step 702 and bacteria taxonomy identification is performed in step 703. In step 704, read counts for each bacterium is generated based on the identified bacteria. The method 700 may include applying a predetermined threshold level for selecting the mapped sequence data in step 702 or after step 702 and prior to step 703. The predetermined threshold level can be determined based on a sequencing method used to generate sequence reads/count output. Having a predetermined threshold level improves the precision for taxonomy identification and thereby resulting in better data robustness that is used for generating the Microbial Index. It will be appreciated that the predetermined threshold level can be determined using well known sequencing methods or techniques according to a desired resolution of the system. The method 700 may further comprise a filtering step after step 702 and before step 703, and the predetermined threshold level can be implemented in the filtering step.

### Obtaining Relative Abundance

The step 722 of obtaining relative abundance may include analyzing the read count abundance in the read count as demonstrated below with reference to the Samples 001 and 002 shown in Table 2 below.

TABLE 2

| | Sample 001 - | Sample 002 - |
|---|---|---|
| Bacteria | Reads Count | Reads Count |
| A (Bacteria of Human | 50 | 10 |
| Health Concern) | | |
| B (Bacteria of Human | 25 | 10 |
| Health Concern) | | |
| C | 100 | 30 |
| D | 100 | 25 |
| E | 75 | 15 |
| F | 25 | 0 |
| G | 115 | 0 |
| Uncharacterized | 10 | 10 |

### Sample 001

Read count abundance of bacteria of human health concern—(A+B)=75
Total read count abundance of all bacteria found on a targeted surface=500
Relative abundance (a percent composition) of bacteria of human health concern=75/500=0.15

### Sample 002

Read count abundance of bacteria of human health concern—(A+B)=20
Total read count abundance of all bacteria found on a targeted surface=100
Relative abundance (a percent composition) of bacteria of human health concern=20/100=0.20
Due to the inherent characteristic of the sequencing-based approaches, the abundance (reads count) are converted to the relative abundance to make such a comparison between Sample 001 and Sample 002.

### Obtaining Relevant Abundance

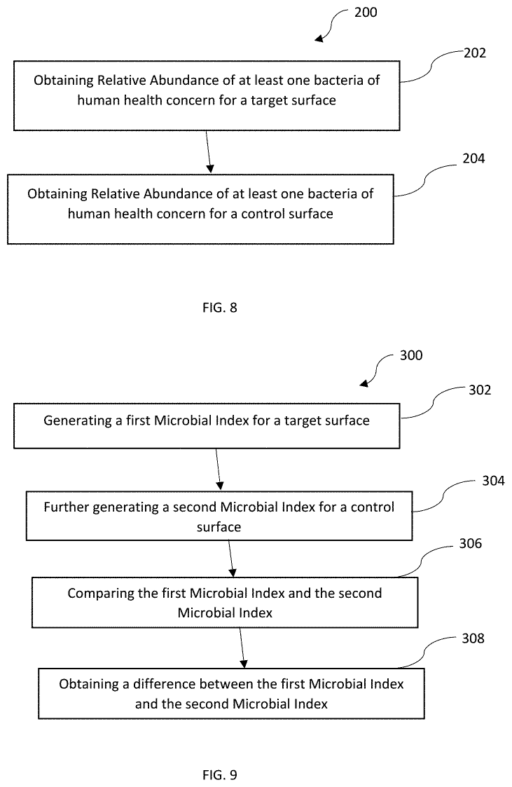

The step 102 of obtaining relative abundance of at least one bacteria of human health concern on at least one surface in the interior space according to the method 101 according to the present invention is described with reference to FIG. 8 which is a flow chart of a process 200 of obtaining relative abundance corresponding to the step 102. The process 200 comprise obtaining a relative abundance of at least one bacteria of human health concern on a target surface in the interior space in step 202. Optionally the process 200 may further comprise obtaining a relative abundance of at least one bacteria of human health concern for a control surface and a relative abundance of all bacteria on the control surface in step 204. While the control surface and the target surface may be in the same interior space, it will be appreciated that this is merely an example, and the target surface and the control surface may be in different interior surfaces depending on a user request.

### Generating Microbial Index

The step 106 of generating the Microbial Index according to the method 101 according to the present invention is described with reference to FIG. 9 which is a flow chart of a process 300 of obtaining relative abundance corresponding to the step 106. The process 300 comprises generating a first Microbial Index for a target surface in step 302 and further generating a second Microbial Index for a control surface in step 304. The first Microbial Index is compared with the second Microbial Index in step 306 and a difference between the first Microbial Index and the second Microbial Index is obtained in step 308. The difference is indicative of a hygiene condition of one of the target surface and the control surface that is better than the other one of the target surface and the control surface, wherein the difference is indicative of a need for improving a hygiene condition of the control surface or the target surface.

Displaying the output 23 may comprise displaying one of: the first Microbial Index, the second Microbial Index, the difference between the first Microbial Index and the second Microbial Index and combinations thereof.

The method 101 may further comprising, prior to the step of obtaining relative abundance of at least one bacteria of human health concern, a step of receiving a user input, wherein the user input comprises a user input request for determining a hygiene condition of an interior space.

### Hygiene Assessment

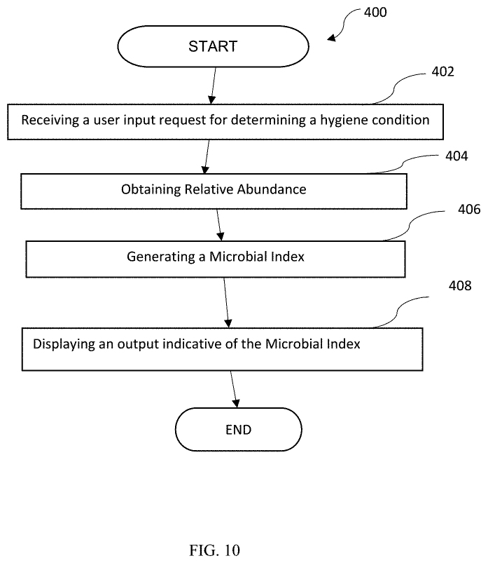

FIG. 10 is a flow chart illustrating a method 400 for providing a hygiene assessment for an interior space according to the present invention.

Referring to FIG. 10, the method 400 comprises receiving a user request for determining a hygiene condition of an interior space in step 402. In step 404, relative abundances of surfaces in the interior space is obtained. A Microbial Index for the interior space is generated in step 406 and an output indicative of the Microbial Index is displayed in step 408.

### Product Recommendation

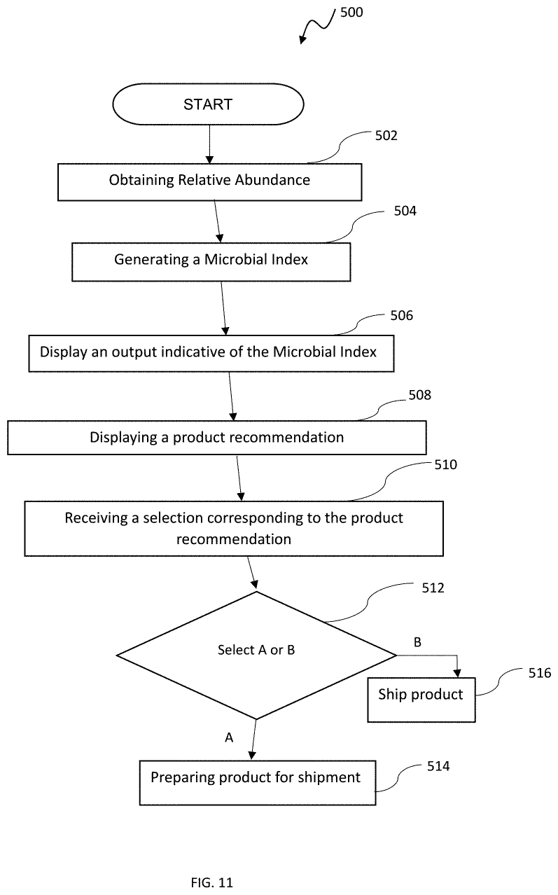

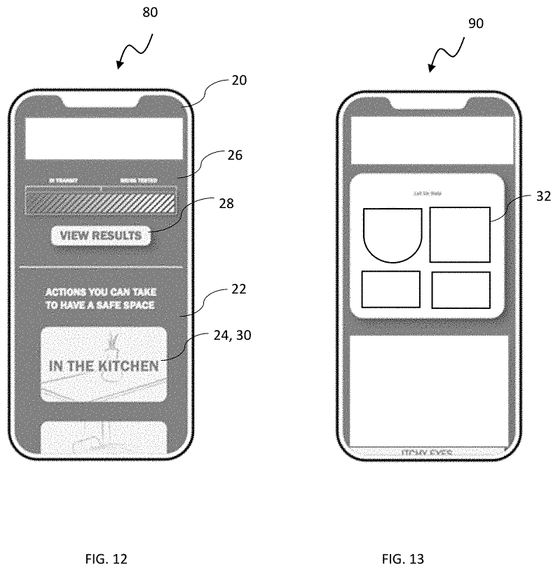

FIG. 11 is a flow chart illustrating a method 500 for providing a product recommendation for improving hygiene condition of an interior space. FIGS. 12 and 13 are screen shots, each illustrating an exemplary user interface cooperating with each other for providing a product recommendation according to the present invention. Although FIGS. 12 and 13 are described as a series of user interfaces which are provided in a sequential manner in response to a preceding user interface, it will be appreciated that the user interfaces of FIGS. 12 and 13 may be programmed in multiple ways to define an overall user interface for providing a product recommendation according to methods according to the present invention as described hereinbefore. The user interfaces of FIGS. 12 to 13 define an exemplary user interface for providing a product recommendation for improving hygiene condition according to the present invention.

Referring to FIG. 11, the method 500 comprises obtaining relative abundance of at least one bacteria of human health concern in step 502. A Microbial Index is generated in step 504. An output indicative of the Microbial Index is displayed in step 506. In step 508, a product recommendation for improving hygiene condition of the interior space is presented to a user, such as for example in a display to the user.

The method 500 may further comprise in or after step 508 of displaying the product recommendation for improving the Microbial Index, a step 510 of receiving a selection corresponding to the product recommendation for improving the Microbial Index.

Optionally, the method 500 may comprise performing at least one of the following based on the selection in step 512: (A) preparing a product for shipment corresponding to the product recommendation in step 514, or (B) shipping the product to a physical address, preferably the product comprises an antimicrobial ingredient in step 516.

The method 500 may further comprise displaying a hygiene improvement plan for the interior space based on the Microbial Index after or in the step 508 of displaying the output.

### Human Machine User Interface

The present invention also relates to a human machine user interface (hereinafter “user interface”) for visualizing a hygiene condition of an interior space. The user interface may be a graphical user interface on a portable electronic apparatus including a touch screen display/display with an input device and an output device.

FIG. 12 illustrates a graphical user interface 80 for visualizing a hygiene condition of an interior space according to the present invention, the graphical user interface 80 being on a portable electronic apparatus including a touch screen display 20. The graphical user interface 80 comprises a first area 22 of the touch screen display 20 displaying an image 24 representative of an interior space. The image 24 may also be a digital image of a physical interior space in which the hygiene condition is determined. There is a second area 26 of the touch screen display 20 different from the first area 24, the second area comprising a first selectable icon 28 for receiving a first user input for displaying a hygiene condition of the interior space. The first area 24 further comprises a second selectable icon 30 superposed on the image 24 for receiving a second user input, wherein a product recommendation 32 (shown in FIG. 13) for improving the hygiene condition is displayed on the touch screen display if the user activates the second selectable icon 30.

FIG. 13 is a screen shot illustrating an exemplary graphical user interface 90 presenting a product recommendation 32 for improving hygiene condition. The product recommendation 32 may be configured as a selectable icon for receiving user input which directs a user to an online e-commerce website for purchasing a consumer product based on the product recommendation.

### Demonstrating of Efficacy of Consumer Product

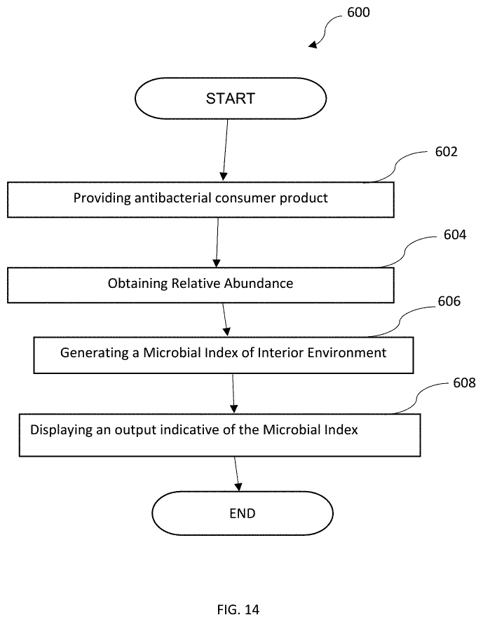

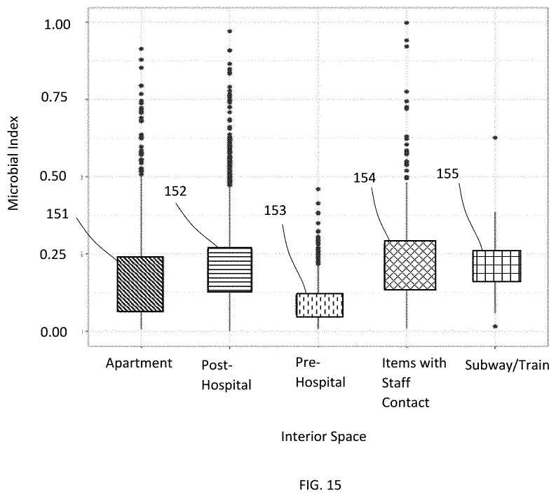

The present invention also relates to a method of demonstrating efficacy of a consumer product for improving a hygiene condition of an interior space. Referring to FIG. 14, the method 600 comprises the steps of the method 101 and differs in that the method 600 further comprises a step 602 of:

providing an antibacterial consumer product in an interior space including an inanimate surface having disposed Klebsiella thereon a bacterium selected from the group consisting of: Staphylococcus aureus, Klebsiella pneumoniae, Proteus mirabilis, Escherichia coli, Enterococcus hirae, Pseudomonas aeruginosa, Salmonella typhi, Salmonella typhimurium, Serratia marcescens, Streptococcus pneumoniae, Streptococcus pyogenes, Vibrio cholerae, Acinetobacter baumannii, Bordetella pertussis, Campylobacter jejuni, Clostridium difficile, Chlamydia pneumoniae, Chlamydia trachomatis, Chlamydia psittaci, Haemophilus influenzae, Helicobacter pylori, Proteus vulgaris.

Providing an antibacterial consumer product in step 602 may be selected from the group consisting of:
exposing the inanimate surface to the product to form a treated inanimate surface;
spraying the inanimate surface with the product to form a treated inanimate surface;
washing the inanimate surface with the product; and combinations thereof.

After step 602, a hygiene condition of the interior space is determined according to the following steps:

Step 604: obtaining relative abundance of at least one bacteria of human health concern from the inanimate surface;
Step 606: generating a Microbial Index of Interior Environment based on the relative abundance;
Step 608: displaying an output indicative of the Microbial Index.

The consumer product may be selected from the group consisting of: air freshener, hard surface cleaning detergent, fabric freshener, hand dishwashing detergent, automatic dishwashing detergent, laundry detergent.

Accordingly, providing the consumer product in step 602 may be for a predetermined period of time before performing steps 604 to 610 based on a time required for the consumer product to be effective against the at least one bacteria of human health concern.

The interior space may be a residential interior environment, a commercial interior environment, a vehicle interior environment or a household appliance interior environment wherein the household appliance is selected from the group consisting of: refrigerator, washing machine, automatic dishwashing machine, air conditioning system.

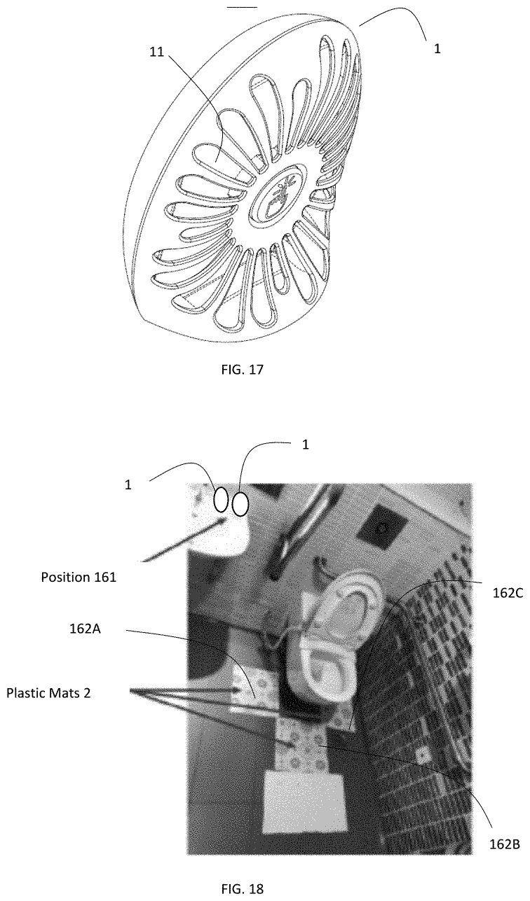

Specifically, the consumer product provided in step 602 may be an exemplary air freshener product 1 as shown in FIG. 17. The product 1 may also comprise a delivery member 11 configured to contain a liquid phase of the composition and allow the liquid phase of the composition to evaporate therefrom. The delivery member may include a wick, a membrane, gel, porous or semi-porous substrate including a felt pad. An exemplary delivery member may be a membrane which is a semi-permeable material which allows some components of matter to pass through but stops other components. Of the components that pass through, the membrane moderates the permeation of components i.e. some components permeate faster than other components. Such components may include molecules, ions or particles.

The air freshener product 1 comprises a membrane 11 having an evaporative surface area of 27 cm² and a freshening composition described in Table 7 under Example 3. However, it is contemplated that the product may be configured for use in a variety of applications to deliver a freshening composition to provide the benefits in interior environments such as furniture for storage of personal items in household and commercial establishments, and the product may include but is not limited to consumer products, such as, for example air freshening products, air fresheners, deodorizers or the like. Therefore, in a different application whereby the interior environment has a different volume such as a shoe cabinet, it will be appreciated that the equipment, materials and methods can be modified accordingly to demonstrate the freshening compositions of the present invention having improved antibacterial efficacy on a surface comprising a permeable material in an interior environment of a different volume. For the purposes of illustrating the present invention in detail, the invention is described below as a non-energized antibacterial air freshening product having a membrane in fluid communication with the composition. However, it will be appreciated that the composition may be delivered from the product to the space through a wick. Further, the product of the present invention can be energized or non-energized.

The membrane 11 may be a microporous membrane and comprise an average pore size of about 0.01 to about 1 microns, about 0.01 to about 0.06 microns, from about 0.01 to about 0.05 microns, about 0.01 to about 0.04 microns, about 0.01 to about 0.03 microns, about 0.02 to about 0.04 microns, or about 0.02 microns. Further, the membrane 11 may be filled with any suitable filler and plasticizer known in the art. Fillers may include finely divided silica, clays, zeolites, carbonates, charcoals, and mixtures thereof. An example of a filled membrane is an ultra-high molecular weight polyethylene (UHMWPE) membrane filled with silica, such as those described in U.S. Pat. No. 7,498,369. Although any suitable fill material and weight percentage may be used, typical fill percentages for silica, may be between about 50% to about 80%, about 60% to about 80%, about 70% to about 80%, or about 70% to about 75% of the total weight of the membrane. Examples of suitable membrane thicknesses include, but are not limited to between about 0.01 mm to about 1 mm, between about 0.1 mm to 0.4 mm, about 0.15 mm to about 0.35 mm, or about 0.25 mm Still further, an evaporative surface area of the membrane 11 may be about 2 cm² to about 100 cm², about 2 cm² to about 25 cm², about 10 cm² to about 50 cm², about 10 cm² to about 45 cm², about 10 cm^{c} to about 35 cm², about 15 cm² to about 40 cm², about 15 cm² to about 35 cm², about 20 cm² to about 35 cm², about 30 cm² to about 35 cm², about 35 cm². The membrane 11 may comprise an evaporative surface area from 2 cm² to 80 cm², from 5 cm² to 54 cm², from 6 cm² to 27 cm², or from 7 cm² to 10 cm².

The air freshener product 1 may be an antibacterial air freshening product comprising a container containing 1 ml to 50 ml of a freshening composition in fluid communication with a delivery member configured to contain a liquid phase of the composition and allow the liquid phase of the composition to evaporate therefrom. The freshening composition may comprise from 0.5% to 20% of a volatile aldehyde mixture, by weight of the composition; wherein the volatile aldehyde mixture consists of:
(i) a C5 to C8 unbranched unsubstituted linear alkenal; and
(ii) a C9 to C14 unbranched unsubstituted linear alkenal, wherein a weight ratio of the C5 to C8 unbranched unsubstituted linear alkenal to the C9 to C14 unbranched unsubstituted linear alkenal is from 3:1 to 1:3.

The following examples are intended to more fully illustrate the present invention and are not to be construed as limitations of the present invention since many variations thereof are possible without departing from the scope of the present invention. All parts, percentages and ratios used herein are expressed as percent weight unless otherwise specified.

### Examples

Test equipment/materials and test compositions are first described under Materials, then Test Methods are provided, and lastly results are discussed. Data is provided demonstrating the compositions of the present invention having improved scent intensity regulation in an interior environment. Equipment and materials used in the Test Methods described hereinafter are listed below.

### Materials

In Examples 1, 2, 3, the following test kit shown in Table 3 below is used for obtaining microbiome samples from surfaces in the interior space for determining hygiene condition thereof.

TABLE 3
Test Kit

| | | Catalog | Storage | Kit Details |
|---|---|---|---|---|
| Manufacturer | Country | number | condition | (if any) |
| HCY technology | China | CY-98000 | Room temp | Swab kit |

### Materials

Details of the microbiome samples evaluated in Examples 1 and 2 are shown in Table 4 below.

TABLE 4

| | Number of | Hypothesize | | |
|---|---|---|---|---|
| Group | samples | Desirable Level | Sites & Surface | Publication |
| Pre-hospital | 357 | High | Room (bedrail, tap, | [5] |
| opening | | | floor); Glove | |
| Household | 670 | High - Medium | Bathroom (door); | [6] |
| | | | Bedroom (floor); | |
| | | | kitchen (counter, | |
| | | | switch, floor) | |
| After opening | 3707 | Medium - Low | Room (bedrail, tap, floor); | [5] |
| of hospital | | | staff station (Armrest, tap, | |
| | | | countertop, tap, floor, phone); | |
| | | | Glove | |
| Subway trains | 73 | Medium - Low | Train (seat, grip, pole, wall); | [7] |
| & stations | | | Station (touchscreen, wall) | |

The corresponding publication references are listed as follows:
[5] Lax et al, Bacterial colonization and succession in a newly opened hospital, Sci Trans Med, 2017
[6] Lax et al, Longitudinal analysis of microbial interaction between humans and the indoor environment, Science, 2014
[7] Hsu et al, Urban transit system microbial communities differ by surface type and Interaction with humans and the environment, mSystems, 2016

### Microbial Index Test Method

### Protocol & Equipment

1) Collection Method (Gloves, sterile flocked swabs, collection tubes, sterile solution of deionized water containing 0.15 M NaCl and 0.1% Tween 20).
   a. Operate with disposable face masks and gloves to prevent contamination.
   b. Take out the disposable swab carefully and open the collecting liquid tube. Then, use a swab to dip in the collecting liquid to make the swab wet.
   c. Place the sample scale card (8 cm×8 cm) properly at the collected indoor items, such as seat cushions, and sampling was performed in the sample scale card. Specifically, wipe the swab while rotating transversely and longitudinally, respectively, for 20 times.
   d. After collection, transfer the disposable swab to the tube containing sample storage liquid immediately, and break the swab along the crease. Then close the lid of the tube and make sure that there is no leakage.
   e. Finally, put the sample preservation solution tube in the biosafety bag.
   f. Storage conditions—(i) before sampling: room temperature 15˜30° C. (ii) after sampling: 2˜8° C.
      2) Bacterial DNA Extraction from Sample.

To extract the DNA from a sample, the sample is thawed. 350 μL of phosphate buffered saline (PBS) is added to the tube containing the sample for extraction. 350 μL of AL buffer solution (from QIAGEN), 40 μL of lysozyme (10 mg/mL), 6 μL of mutanolysin (25000U), and 300 mg of glass beads are added to the tube. The contents of the tube are mixed by vortexing. The tube is then incubated at 37° C. for one hour. The tube is then transferred to a tissue grinder (supplied by QIAGEN) and processed for 3 minutes at 26 Hz. 20 μL of protease K (from QIAGEN reagent kit) is added to the tube, then the tube is capped and shaken until homogeneous. The tube is then incubated at 56° C. for 3 hours.

The supernatant from the tube is then transferred to a new, clean tube and the swab is discarded. The beads are washed twice with 200 μL of distilled water. A ½ volume of alcohol is added to the tube and the contents are mixed until they become homogenous. Load the tube contents into a DNeasy centrifugation chromatographic column (purchased from QIAGEN) which has been placed in a clean centrifuge tube and allow it to be absorbed. Centrifuge the column at 8000 rpm/min for one minute. Discard the waste liquid which comes through the column and the centrifuge tube.

Place the chromatographic column into a clean centrifuge tube. Add 500 μL of AW1 buffer (QIAGEN), allow it to absorb into the column, and then centrifuge the column at 8000 rpm/min for one minute. Discard the waste liquid and the collection tube. Once again, place the chromatographic column into a clean centrifuge tube. Add 500 μL of AW2 buffer, allow it to absorb into the column, and then centrifuge the column at 14000 rpm/min for 3 minutes. Discard the waste liquid and the collection tube. Allow the chromatographic column to dry at room temperature. A new column is used with each sample.

### 3) Sequencing Methods

16S rRNA sequencing approach—The microbiota of the extracted DNA from the surface samples can be determined by putting it through the 16S rRNA sequencing method as known. The sequencing can be done on a target region and with a selected primer and the regions targeted are V3-V4. In addition, the sequencing can be done by utilizing a reagent kit (Illumina Miseq 250/300). A 20 μL reaction mixture is made by combining 10 μL of Sybr green, 0.5 μL of upstream primer, 0.5 μL of downstream primer, 5 μL of deionized H₂O 5 and 4 μL of the extracted DNA. The reaction system is then placed into a 96-well plate. The 96-well plate is placed into a real-time fluorescent quantitative PCR device for reaction, including pre-denaturation at 94° C. for 10 mM, denaturation at 94° C. for 30 s, annealing at a suitable annealing temperature for 30s, extension at 72° C. for 45s, for 45 cycles; and lastly, extension at 72° C. for 10 min. Once this is complete, the number of copies of genes of the various genera of bacteria in the samples can be calculated. In combination with the amplification curve of the standard sample, the relative abundance of the various strains in samples can be obtained.

2b-RAD sequencing approach—Library preparation begins with the digestion of 1 pg-200 ng genomic DNA in a 15-μl reaction using 4 U BcgI (NEB) at 37° C. for 3 h. A small aliquot (˜30 ng) is separated on a 1% agarose gel to verify digestion. Next, 12 μl of a ligation master mix containing 0.2 μM library-specific adaptors (slx-ada1 and slx-ada2), 1 mM ATP (NEB), and 800 U T4 DNA ligase (NEB) is added to the digestion product and incubated for 16 h (4° C. for BcgI digests). Then heat inactivation is performed for BcgI at 65° C. for 20 min. Ligation products are amplified in three 20-μl reactions per sample, each composed of 7 μl ligated DNA, 0.1 μM each primer (slx-p1 and slx-p2 for Illumina), 0.3 mM dNTP, 1× Phusion HF buffer and 0.4 U Phusion high fidelity DNA polymerase (NEB). PCR is conducted in a DNA Engine Tetrad 2 thermal cycler (Bio-Rad) with 20-22 cycles of 98° C. for 5 s, 60° C. for 20 s and 72° C. for 10 s and then a final extension of 10 min at 72° C. The target band (Illumina: 96 bp) is excised from a 2% agarose gel, and the DNA is allowed to diffuse from the agarose into nuclease-free water for 12 h at 4° C. Finally, barcodes are introduced by means of PCR with platform-specific barcode-bearing primers. Each 20 μl PCR reaction contains 25 ng of gel-extracted PCR product, 0.1 μM of each primer (slx-p1 and slx-p3 for Illumina), 0.3 mM dNTP, 1× Phusion HF buffer and 0.4 U Phusion high-fidelity DNA polymerase; four or five cycles of the PCR profile listed above are performed. PCR products are purified using QIAquick PCR purification kit (Qiagen) before sequencing. Illumina sequencing (xTen) is performed at the Qingdao OE BioTech.

### 4) Microbiome Profiling

16S rRNA—Trimmomatic is used for reads quality control (QC). FLASH is used for the merger of the sequence data of the two ends of the sequence. Fastx Toolkit is used to carry out a second quality control. The main parameters in the process include: Trimmomatic: SLIDINGWINDOW:30:25MINLEN:25; FLASH:-M 200 -m 5 -x 0.1; Fastx Toolkit: -Q 33 -q 25 -p 80. After QC, the reads are mapped against the V3-V4 hypervariable sequence from the NBCI 16S rRNA Refseq database using blastn v2.6.0+ for taxonomy identification & classification at 99.75% similarity level with alignment length >400 bp.

2b-RADm—Species identification is performed, each of the sequenced 2B tags after quality control will be searched (using built-in Perl script) against the unique 2B tag database which contains all unique 2B tags theoretically inferred from more than 30,000 microbial species genomes in NBBI RefSeq database. G score is used as a threshold (default G is 5) to control the false-positive identification with the formula below.

G score_{species i}=\sqrt{Sᵢ×tᵢ}

S: Number of sequenced unique 2b tags belongs to species i.

t: Number of theoretical 2b tags in species i that actually been sequenced.

Then the sequenced tags will be reused to search against an automatically generated 2b unique tag database which only contains the genomes of identified species in last step. The number of newly defined unique 2b tags will be counted, and then divided by the number of theoretical 2b tag number of this species (mean theoretical 2b tags number if multi strains are detected in this species), generating the relative abundance of this species (see below formula).

$$Relative {abundance}_{species i} = \frac{{S}_{i} / {T}_{i}}{{\sum }_{i = 1}^{n} {S}_{i} / {T}_{i}}$$

S: Number of sequenced unique 2b tags belongs to species i.

T: Number of theoretical 2b tags in species i.

### 5) Microbial Index of Interior Environment Tabulation

$$Microbial Index of Interior Environment = \frac{{\sum }_{i = 1}^{N} {P}_{i}}{{\sum }_{i = 1}^{M} {X}_{i}}$$

N: Number of bacteria of human health concern identified in a microbial community in the interior space;
P: Relative abundance of i-th bacteria of human health concern in the microbial community;
M: Number of all bacteria identified in the microbial community
X: Relative abundance of i-th bacteria identified in the microbial community

The number of potential bacteria species of human health concern detected and tabulated for (i) 16-rRNA approach is 173 and (ii) 2-RAD approach is 299. This list of potential bacteria pathogenic species is curated from Miao et al, BMC Bioinforatics, 2017 (https://bmcbioinformatics.biomedcentral.com/articles/10.1186/s12859-017-1975-3) and recognized by Chinese Center for Disease Control and Prevention (CDC) (http://16spip.mypathogen.cn/). Total bacterial load is based on the total plate count of bacteria as colony forming unit (CFU).

### Example 1

FIG. 15 is a graph illustrating a Microbial Index for different interior spaces, wherein each of the different interior spaces is a built interior space, i.e. an apartment. Based on microbiome samples obtained from surfaces in the apartment, and analyzing the microbiome samples with 16S rRNA sequencing method (n=4807, wherein n is the total number of bacteria in each of the microbiome samples obtained from the apartment), the apartment has a Microbial Index of 0.25. The Microbial Index of the apartment is generated based on steps described under Microbial Test Method described hereinbefore.

The relative abundances of the microbiome samples for generating the Microbial Indices are obtained from the above described publications [5] Lax et al, Bacterial colonization and succession in a newly opened hospital, Sci Trans Med, 2017, [6] Lax et al, Longitudinal analysis of microbial interaction between humans and the indoor environment, Science, 2014, [7] Hsu et al, Urban transit system microbial communities differ by surface type and Interaction with humans and the environment., mSystems, 2016.

A summary of the Microbial indices corresponding to FIG. 15 is shown in Table 5 below.

TABLE 5

| Interior Space | Microbial Index | Output |
|---|---|---|
| Apartment | 0.142 | Feature 151 |
| Post opening hospital | 0.199 | Feature 152 |
| Pre opening hospital | 0.076 | Feature 153 |
| Staff samples collected | 0.211 | Feature 154 |
| from staff-associated | | |
| surfaces (example - shoe | | |
| surface, personal cell | | |
| phone, shirt hem, hospital pager) | | |
| Subway_Station | 0.209 | Feature 155 |

### Example 2

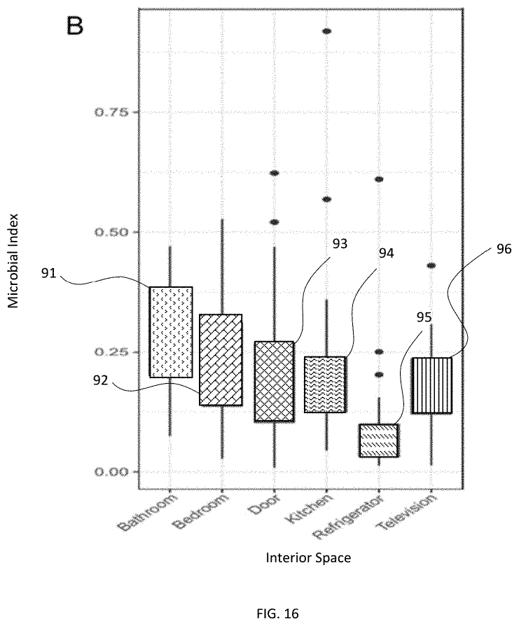

FIG. 16 is a graph illustrating different Microbial indices for a plurality of interior spaces in a home environment. The plurality of interior spaces may include bathroom, bedroom, door, kitchen, refrigerator. Microbial indices of each of the plurality of interior spaces in the home environment may be generated according to the steps described under Microbial Index Test Method described hereinbefore. A summary of the Microbial indices corresponding to FIG. 16 is shown in Table 6 below. The relative abundances of the microbiome samples for generating the Microbial Indices are obtained from Lax et al, Longitudinal analysis of microbial interaction between humans and the indoor environment, Science, 2014.

TABLE 6

| Interior Space | Microbial Index | Output |
|---|---|---|
| Bathroom | 0.30 | Feature 91 |
| Bedroom | 0.23 | Feature 92 |
| Door | 0.18 | Feature 93 |
| Kitchen | 0.19 | Feature 94 |
| Refrigerator | 0.05 | Feature 95 |
| Television | 0.16 | Feature 96 |

### Example 3

In Example 3, an air freshening product evaluated is designed as a consumer product, such as a toilet deodorizer, for evaporating a freshening composition in a toilet to deliver a variety of benefits such as bacteria growth prevention on permeable inanimate surfaces, freshening, malodor removal or scenting of air in the toilet. The size of the interior space evaluated in Example 3 is 7.2 m², and two units of an exemplary air freshener product 1 as shown in FIG. 17 is evaluated in Example 3. The air freshener product 1 comprises a membrane having an evaporative surface area of 27 cm² and a freshening composition described in Table 7 below.

Table 7 describes the freshening composition which is evaluated in Example 3. The freshening composition contains a volatile aldehyde mixture of a C5 to C8 unbranched unsubstituted linear alkenal ((E)-2-Hexen-1-al CAS No. 6728-26-3 as an example) and a C9 to C14 unbranched unsubstituted linear alkenal ((E)-2-decen-1-al CAS No. 3913-81-3 as an example) in a weight ratio of 1:1.

TABLE 7
Formulations of Freshening Composition

| | |
|---|---|
| Ingredients (by weight of the composition (wt %) | Freshening |

| CAS No. | IUPAC Name | Composition |
|---|---|---|
| 6728-26-3 | (E)-2-Hexen-1-al | 3% |
| 3913-81-3 | (E)-2-Decen-1-al | 3% |
| — | Perfume Accord 1* | 94% |

| | |
|---|---|
| Weight Percentage Total: | 100 |

*Accord ingredients are not disclosed by the manufacturer.

The product is evaluated according to a placement as shown in FIG. 18. Specifically, to simulate a high usage toilet at home, 10 male test subjects are recruited to urinate in an interior space configured to simulate the environment of a bathroom containing a toilet (hereinafter “toilet”). They are requested to use this toilet for a minimum of 3 times per day for 2 periods of time (“legs”), each period of time comprising 5 consecutive days.

In the first leg of 5 days, the toilet does not have any product. In the second leg of 5 days, samples of an air freshener product 1 having the freshening composition of Table 7 are placed in the toilet. Specifically, two units of air freshener product 1 (each unit having 6.5 ml of the freshening composition) are placed on the basin in the toilet (See Position 161 of FIG. 18). New plastic mats 2 are placed at three sides of the toilet bowl for each leg of the study (See Positions 162A, 162B, 162C). The plastic mats 2 are not cleaned/removed during each leg usage.

The test subjects are required to wear shoe cover during the usage of the toilet and should not cover the toilet bowl when flushing.

Microbiome samples (three replicates) are collected from each plastic mat for total bacteria load and microbiome measurement according to the Microbial Test Method described herein before using the 16S rRNA sequencing method described hereinbefore to generate raw sequencing data for analysis and generation of the Microbial Index.

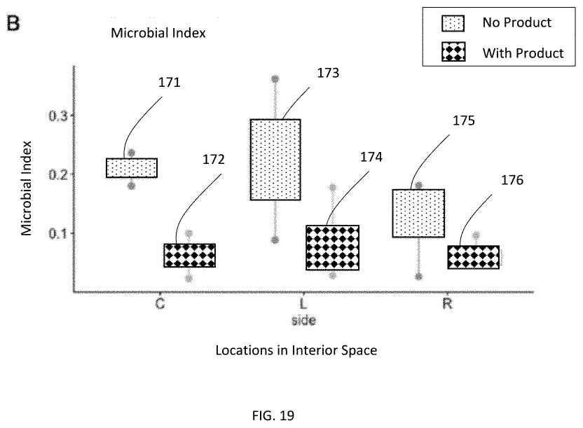

FIG. 19 is a graph illustrating Microbial indices obtained from the plastic mats 2 in both legs under Microbial Index Test Method described hereinbefore. A summary of the Microbial indices corresponding to FIG. 19 is shown in Table 8 below.

TABLE 8

| Interior Space | Microbial Index | Output Feature |
|---|---|---|
| Position 162B (Center) - | 0.21 | Feature 171 |
| First Plastic Mat 2 without | | |
| Air Freshener Product | | |
| Position 162B (Center) - | 0.06 | Feature 172 |
| Second Plastic Mat 2 with | | |
| Air Freshener Product | | |
| Position 162A (Left) | 0.22 | Feature 173 |
| Third Plastic Mat 2 without | | |
| Air Freshener Product | | |
| Position 162A (Left) | 0.05 | Feature 174 |
| Third Plastic Mat 2 with | | |
| Air Freshener Product | | |
| Position 162C (Right) - | 0.16 | Feature 175 |
| Fourth Plastic Mat 2 without | | |
| Air Freshener Product | | |
| Position 162C (Right) - | 0.05 | Feature 176 |
| Fourth Plastic Mat 2 with | | |
| Air Freshener Product | | |

The above results show that providing an air freshener product 1 having a composition of Table 7 reduces a Microbial Index in each of the plurality of surfaces (floor mats) thereby improving an overall hygiene condition in the toilet.

Representative embodiments of the present disclosure described above can be described as set out in the following paragraphs:

### Combinations

A. A computer-implemented method for determining a hygiene condition of an interior space, the method comprising the steps of:
a) obtaining relative abundance of at least one bacterium of human health concern in an interior space;
b) generating a Microbial Index of Interior Space (“Microbial Index”) based on the relative abundance; and
c) displaying an output indicative of a hygiene condition of the interior space based on the Microbial Index;

Wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in the interior space;
P: Relative abundance of i-th bacteria of human health concern in the microbial community.
   B. The method of B, wherein obtaining the relative abundance comprises:
i) obtaining relative abundance of at least one bacteria of human health concern on a target surface in the interior space; and
ii) o optionally obtaining relative abundance of at least one bacteria of human health concern for a control surface and relative abundance of all bacteria on the control surface;

wherein the control surface is in the same interior space as the target surface or a different interior space from the target surface.

C. The method of A or B, wherein generating the Microbial Index comprises
iii) generating a first Microbial Index for a target surface;
iv) further generating a second Microbial Index for a control surface;
v) comparing the first Microbial Index with the second Microbial Index;
vi) obtaining a difference between the first Microbial Index and the second Microbial Index; wherein the difference is indicative of a hygiene condition of one of the target surface and the control surface that is better than the other one of the target surface and the control surface, wherein the difference is indicative of a need for improving a hygiene condition of the control surface or the target surface.
   D. The method of C, wherein displaying the output comprises displaying one of: the first Microbial Index, the second Microbial Index, the difference between the first Microbial Index and the second Microbial Index and combinations thereof.
   E. The method according to any one of A to D, wherein the function has the following formula:

$$Microbial Index of Interior Space = \frac{{\sum }_{i = 1}^{N} {P}_{i}}{{\sum }_{i = 1}^{M} {X}_{i}}$$

N: Number of bacteria of human health concern identified in a microbial community in the interior space;
P: Relative abundance of i-th bacteria of human health concern;
M: Number of all bacteria identified in the microbial community;
X: Relative abundance of i-th bacteria identified in the microbial community.
   F. The method according to any one of A to E, further comprising analyzing (721) raw sequence data of all bacteria in the microbial community prior to obtaining relative abundance; wherein relative abundance is obtained based on the analyzed raw sequence data.
   G. The method according to F, wherein analyzing raw sequence data comprises:

receiving raw sequence data of bacteria in the microbial community;

mapping raw sequence data of bacteria in the microbial community against content in a Microbial Database;

identifying taxonomy of bacteria in the microbial community based on the mapped raw sequence data; and

generating read counts for each bacterium based on the identified bacteria.

H. The method according to any one of A to G, further comprising, prior to step (a), receiving a user input, wherein the user input comprises a request for determining a hygiene condition of an interior space.
I. The method according to any one of A to H, further comprising in or after step (d) of displaying the output, the steps of:

displaying a product recommendation for improving the Microbial Index;

receiving a selection corresponding to the product recommendation for improving the Microbial Index; and

optionally, performing at least one of the following based on the selection: (A) preparing a product for shipment corresponding to the product recommendation, or (B) shipping the product to a physical address, wherein the product comprises an antimicrobial ingredient

J. The method according to any one of A to I, further comprising displaying a hygiene improvement plan for the interior space based on the Microbial Index after or in the step (c) of displaying the output.
K. The method according to any one of A to J, wherein the interior space is a residential interior environment, a commercial interior environment, a vehicle interior environment or a household appliance interior environment, wherein the household appliance is selected from the group consisting of: refrigerator, washing machine, automatic dishwashing machine, air conditioning system.
L. The method according to any one of A to J, wherein obtaining the relative abundance comprises obtaining the relative abundance of at least one bacteria of human health concern on at least one surface in the interior space.
M. The method according to L, wherein the at least one surface is an inanimate surface selected from a group consisting of: vehicle interior surfaces, fabrics, built environment surfaces, household surfaces, wherein the household surface is selected from a group consisting of: floors, walls, carpet padding, towels, carpets.
N. A system for providing a hygiene assessment for an interior space over a network to a portable electronic device, the system and the device are connected to the network, the system comprising:

a web application capable of being compiled to run on a server computing system for receiving a user input request from a portable electronic device for determining a hygiene condition of an interior space, wherein said server computing system is in communication with a sequencing content analysis server configured to store the received user input request;

wherein the server computing system comprises:

a server in communication with the web application through a network, wherein said server comprises a processor configured to, based on computer-executable instructions stored in a memory to:

analyze raw sequencing data of all bacteria in a microbial community in the interior space;

obtain a relative abundance of at least one bacterium of human health concern in the interior space; and

generate a Microbial Index of Interior Environment (“Microbial Index”) indicative of the hygiene condition of the interior space based on the relative abundance; and

a display generating unit in communication with the server computing system, configured to display an output indicative of the Microbial Index for the interior space on a portable electronic device (78) connected to the server through the network; wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in an interior space;
P: Relative abundance of i-th bacteria of human health concern in the microbial community.
   O. A system for determining a hygiene condition of an interior space, the system comprising:

a mobile application capable of being compiled to run on a client computing system for receiving a user input request for determining a hygiene condition of an interior space, wherein said computing system is in communication with a content server configured to store the obtained user input request;

a server different from the content server in communication with the mobile application through a network; wherein said server comprises a processor configured to, based on computer-executable instructions stored in a memory to:

obtain a relative abundance of at least one bacterium of human health concern obtained from the interior space; and

generate a Microbial Index of Interior Environment (“Microbial Index”) indicative of the hygiene condition of the interior space based on the relative abundance; and

an output device in communication with the client computing system and the server, configured to display an output indicative of the Microbial Index for the interior space; wherein the Microbial Index is characterized by a function of the relative abundance of the at least one bacterium of human health concern defined by F(Σᵢ₌₁^{N}Pᵢ), wherein

N: Number of bacteria of human health concern identified in a microbial community in the interior space;

P: Relative abundance of i-th bacteria of human health concern in the microbial community.

P. The system of N or O further comprising:

receiving a selection corresponding to a product recommendation for improving the Microbial Index; and

performing at least one of the following based on the selection: (1) preparing a product for shipment corresponding to the product recommendation, or (2) shipping the product to a physical address, wherein the product comprises an antimicrobial ingredient.

Q. The system of any one of N to P, wherein the server is configured to generate a hygiene improvement plan for the interior space based on the Microbial Index.
R. The system of any one of N to Q, wherein the user input is associated with a user account, wherein the user input comprises a user input request for a test kit for collecting microbiome from at least one surface in the interior space.
S. The system of any one of N to R, wherein the interior space is a residential interior environment, a commercial interior environment, a vehicle interior environment or a household appliance interior environment, wherein the household appliance is selected from the group consisting of: refrigerator, washing machine, automatic dishwashing machine, air conditioning system; wherein the test kit comprises instructions for collecting microbiome from the at least one surface in the interior space.
T. A graphical user interface for visualizing a hygiene condition of an interior space, the graphical user interface being on a portable electronic apparatus including a touch screen display with an input device and an image obtaining device, the graphical user interface comprising:

a first area of the touch screen display displaying an image representative of an interior space;

a second area of the touch screen display different from the first area, the second area comprising a first selectable icon for receiving a first user input for displaying a hygiene condition of the interior space; and

wherein the first area comprises a second selectable icon superposed on the image for receiving a second user input, wherein a product recommendation for improving the hygiene condition is displayed on the touch screen display if the user activates the second selectable icon.

U. A method of demonstrating efficacy of a consumer product for improving a hygiene condition of an interior space, the method comprising the steps of:

providing at least one antibacterial consumer product in an interior space including an inanimate surface having disposed thereon a bacterium selected from the group consisting of: Staphylococcus aureus, Klebsiella pneumoniae, Proteus mirabilis, Escherichia coli, Enterococcus hirae, Pseudomonas aeruginosa, Salmonella typhi, Salmonella typhimurium, Serratia marcescens, Streptococcus pneumoniae, Streptococcus pyogenes, Vibrio cholerae, Acinetobacter baumannii, Bordetella pertussis, Campylobacter jejuni, Clostridium difficile, Chlamydia pneumoniae, Chlamydia trachomatis, Chlamydia psittaci, Haemophilus influenzae, Helicobacter pylori, Proteus vulgaris; and

determining a hygiene condition of the interior space based on a method according to any one of A to L.

V. The method of U, wherein providing the at least one antibacterial consumer product is selected from the group consisting of:

exposing the inanimate surface to the product to form a treated inanimate surface;

spraying the inanimate surface with the product to form a treated inanimate surface;

washing the inanimate surface with the product; and combinations thereof.

W. The method of any one of U to V, wherein the consumer product is selected from the group consisting of: air freshener, hard surface cleaning detergent, fabric freshener, hand dishwashing detergent, automatic dishwashing detergent, laundry detergent.
X. The method of any one of claims U to W, providing at least one antibacterial air freshener in the interior space, wherein the antibacterial air freshening product comprises:

a container containing 1 ml to 50 ml of a freshening composition in fluid communication with a delivery member configured to contain a liquid phase of the composition and allow the liquid phase of the composition to evaporate therefrom;

wherein the composition comprises:

from 0.5% to 20% of a volatile aldehyde mixture, by weight of the composition; wherein the volatile aldehyde mixture consists of:
(iii) A C5 to C8 unbranched unsubstituted linear alkenal; and
(iv) A C9 to C14 unbranched unsubstituted linear alkenal, wherein a weight ratio of the C5 to C8 unbranched unsubstituted linear alkenal to the C9 to C14 unbranched unsubstituted linear alkenal is from 3:1 to 1:3
   Y. The method according to any one of claims U to X, wherein the interior space is a residential interior environment, a commercial interior environment, a vehicle interior environment or a household appliance interior environment wherein the household appliance is selected from the group consisting of: refrigerator, washing machine, automatic dishwashing machine, air conditioning system.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A computer-implemented method for determining a hygiene condition of an interior space, the method comprising the steps of:
(a) obtaining relative abundance of at least one bacterium of human health concern in an interior space;
(b) generating a Microbial Index of Interior Space (“Microbial Index”) based on the relative abundance; and
(c) displaying an output indicative of a hygiene condition of the interior space based on the Microbial Index;
wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in the interior space;
P: Relative abundance of i-th bacteria of human health concern in the microbial community.

2. The method of claim 1, wherein obtaining the relative abundance comprises:
i) obtaining relative abundance of at least one bacteria of human health concern on a target surface in the interior space; and
ii) optionally obtaining relative abundance of at least one bacteria of human health concern for a control surface and relative abundance of all bacteria on the control surface; wherein the control surface is in the same interior space as the target surface or a different interior space from the target surface.

3. The method of claim 1, wherein generating the Microbial Index comprises
iii) generating a first Microbial Index for a target surface;
iv) further generating a second Microbial Index for a control surface;
v) comparing the first Microbial Index with the second Microbial Index;
vi) obtaining a difference between the first Microbial Index and the second Microbial Index; wherein the difference is indicative of a hygiene condition of one of the target surface and the control surface that is better than the other one of the target surface and the control surface, wherein the difference is indicative of a need for improving a hygiene condition of the control surface or the target surface.

4. The method of claim 3, wherein displaying the output comprises displaying one of: the first Microbial Index, the second Microbial Index, the difference between the first Microbial Index and the second Microbial Index and combinations thereof.

5. The method according to claim 1, wherein the function has the following formula:
$$Microbial Index of Interior Space = \frac{{\sum }_{i = 1}^{N} {P}_{i}}{{\sum }_{i = 1}^{M} {X}_{i}}$$
N: Number of bacteria of human health concern identified in a microbial community in the interior space;
P: Relative abundance of i-th bacteria of human health concern;
M: Number of all bacteria identified in the microbial community;
X: Relative abundance of i-th bacteria identified in the microbial community.

6. The method according to claim 1, further comprising analyzing raw sequence data of all bacteria in the microbial community prior to obtaining relative abundance; wherein relative abundance is obtained based on the analyzed raw sequence data.

7. The method according to claim 6, wherein analyzing raw sequence data comprises:
receiving raw sequence data of bacteria in the microbial community;
mapping raw sequence data of bacteria in the microbial community against content in a Microbial Database;
identifying taxonomy of bacteria in the microbial community based on the mapped raw sequence data; and
generating read counts for each bacterium based on the identified bacteria.

8. The method according to claim 1, further comprising, prior to step (a), receiving a user input, wherein the user input comprises a request for determining a hygiene condition of an interior space.

9. The method according to claim 1, further comprising in or after step (d) of displaying the output, the steps of:
displaying a product recommendation for improving the Microbial Index;
receiving a selection corresponding to the product recommendation for improving the Microbial Index; and
optionally, performing at least one of the following based on the selection: (A) preparing a product for shipment corresponding to the product recommendation, or (B) shipping the product to a physical address, wherein the product comprises an antimicrobial ingredient.

10. The method according to claim 1, further comprising displaying a hygiene improvement plan for the interior space based on the Microbial Index after or in the step (c) of displaying the output.

11. The method according to claim 1, wherein the interior space is a residential interior environment, a commercial interior environment, a vehicle interior environment or a household appliance interior environment, preferably the household appliance is selected from the group consisting of: refrigerator, washing machine, automatic dishwashing machine, air conditioning system.

12. The method according to claim 1, wherein obtaining the relative abundance comprises obtaining the relative abundance of at least one bacteria of human health concern on at least one surface in the interior space.

13. The method according to claim 12, wherein the at least one surface is an inanimate surface selected from a group consisting of: vehicle interior surfaces, fabrics, built environment surfaces, household surfaces, preferably the household surface is selected from a group consisting of: floors, walls, carpet padding, towels, carpets.

14. A system for providing a hygiene assessment for an interior space over a network to a portable electronic device, the system and the device are connected to the network, the system comprising:
a web application capable of being compiled to run on a server computing system for receiving a user input request from a portable electronic device for determining a hygiene condition of an interior space, wherein said server computing system is in communication with a sequencing content analysis server configured to store the received user input request;
wherein the server computing system comprises:
a server in communication with the web application through a network,
wherein said server comprises a processor configured to, based on computer-executable instructions stored in a memory to:
analyze raw sequencing data of all bacteria in a microbial community in the interior space;
obtain a relative abundance of at least one bacterium of human health concern in the interior space; and
generate a Microbial Index of Interior Environment (“Microbial Index”) indicative of the hygiene condition of the interior space based on the relative abundance; and
a display generating unit in communication with the server computing system, configured to display an output indicative of the Microbial Index for the interior space on a portable electronic device connected to the server through the network; wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in an interior space;
P: Relative abundance of i-th bacteria of human health concern in the microbial community.

15. A system for determining a hygiene condition of an interior space, the system comprising:
a mobile application capable of being compiled to run on a client computing system for receiving a user input request for determining a hygiene condition of an interior space, wherein said computing system is in communication with a content server configured to store the obtained user input request;
a server different from the content server in communication with the mobile application through a network; wherein said server comprises a processor configured to, based on computer-executable instructions stored in a memory to:
obtain a relative abundance of at least one bacterium of human health concern obtained from the interior space; and
generate a Microbial Index of Interior Environment (“Microbial Index”) indicative of the hygiene condition of the interior space based on the relative abundance; and
an output device in communication with the client computing system and the server, configured to display an output indicative of the Microbial Index for the interior space; wherein the Microbial Index is characterized by a function of the relative abundance of the at least one bacterium of human health concern defined by F(Σᵢ₌₁^{N}Pᵢ), wherein
N: Number of bacteria of human health concern identified in a microbial community in the interior space;
P: Relative abundance of i-th bacteria of human health concern in the microbial community.

16. The system of claim 14 further comprising:
receiving a selection corresponding to a product recommendation for improving the Microbial Index; and
performing at least one of the following based on the selection: (1) preparing a product for shipment corresponding to the product recommendation, or (2) shipping the product to a physical address, preferably the product comprises an antimicrobial ingredient.

17. The system of claim 14, wherein the server is configured to generate a hygiene improvement plan for the interior space based on the Microbial Index.

18. The system of claim 14, wherein the user input is associated with a user account, preferably the user input comprises a user input request for a test kit for collecting microbiome from at least one surface in the interior space.

19. The system of claim 14, wherein the interior space is a residential interior environment, a commercial interior environment, a vehicle interior environment or a household appliance interior environment, preferably the household appliance is selected from the group consisting of: refrigerator, washing machine, automatic dishwashing machine, air conditioning system; preferably the test kit comprises instructions for collecting microbiome from the at least one surface in the interior space.

20. A method of demonstrating efficacy of a consumer product for improving a hygiene condition of an interior space, the method comprising the steps of:
providing at least one antibacterial consumer product in an interior space including an inanimate surface having disposed thereon a bacterium selected from the group consisting of: Staphylococcus aureus, Klebsiella pneumoniae, Proteus mirabilis, Escherichia coli, Enterococcus hirae, Pseudomonas aeruginosa, Salmonella typhi, Salmonella typhimurium, Serratia marcescens, Streptococcus pneumoniae, Streptococcus pyogenes, Vibrio cholerae, Acinetobacter baumannii, Bordetella pertussis, Campylobacter jejuni, Clostridium difficile, Chlamydia pneumoniae, Chlamydia trachomatis, Chlamydia psittaci, Haemophilus influenzae, Helicobacter pylori, Proteus vulgaris; and
determining a hygiene condition of the interior space based on a method according to claim 1.
